(19) United States
(12) Patent Application Publication
(10) Pub. No.: US 2023/0398370 A1
(43) Pub. Date: Dec. 14, 2023

(54) BATHROOM SYSTEM FOR VITAMIN D SYNTHESIS AND OPERATING METHOD THEREOF

(71) Applicant: CEBIEN CO., LTD., Gyeonggi-do (KR)

(72) Inventors: Bae-Dong JHIN, Gyeonggi-do (KR); Byeong-Seol GO, Gyeonggi-do (KR); In Shik RYU, Gyeonggi-do (KR)

(21) Appl. No.: 18/035,182

(22) PCT Filed: Nov. 24, 2020
(86) PCT No.: PCT/KR2020/016701

§ 371 Date: May 3, 2023

(30) Foreign Application Priority Data
Nov. 6, 2020 (KR) 10-2020-0147661

(51) Int. Cl. A61N 5/06

(52) U.S. Cl. CPC A61N 5/0613; A61N 2005/0626; A61N 2005/0661; A61N 2005/0632; A61N 2005/0668

(57) ABSTRACT

A bathroom system for vitamin D synthesis includes an environmental information collection unit which collects external environment information regarding the outdoors, a user information collection unit which collects user activity information about a user's outdoor activities, a data analysis unit which analyzes the external environment information and the user activity information to derive the user's predicted amount of vitamin D synthesis, and compares the predicted amount of vitamin D synthesis with a preset reference recommended amount of vitamin D, to derive an additional amount of vitamin D synthesis, a light source for vitamin synthesis provided in a bathroom facility to irradiate light for synthesis of vitamin D in the user's body, and a light source control unit which controls the light source for vitamin synthesis according to the additional amount of vitamin D synthesis, which has been derived from the data analysis unit.

### CROSS REFERENCE TO RELATED APPLICATIONS AND CLAIM OF PRIORITY

This application claims benefit under 35 U.S.C. 119, 120, 121, or 365(c), and is a National Stage entry from International Application No. PCT/KR2020/016701, filed Nov. 24, 2020, which claims priority to the benefit of Korean Patent Application No. 10-2020-0147661 filed in the Korean Intellectual Property Office on Nov. 6, 2020, the entire contents of which are incorporated herein by reference.

### BACKGROUND

### 1. Technical Field

The present disclosure relates to a bathroom system for vitamin D synthesis and an operating method thereof, and more particularly, to a bathroom system for vitamin D synthesis that derives an additional amount of vitamin D synthesis additionally required for a user and induces vitamin D synthesis in the user through a light source for vitamin synthesis provided in bathroom facilities and an operating method thereof.

### 2. Background Art

Vitamin D (calciferol) that is an essential nutrient needed to maintain health is divided into D₂ (ergocalciferol) and D₃ (cholecalciferol). Vitamin D₂ is produced from ergosterol that is a sterol in yeasts and plants, and vitamin D₃ is produced from the precursor of cholesterol, 7-Dehydrocholesterol, when the skin is exposed to ultraviolet rays in sunlight.

Vitamin D is synthesized in skin cells from cholesterol derivatives (7-hydrocholesterol) after exposure to sunlight and plays a crucial role in strengthening bones by increasing calcium absorption from the small intestine. It is known that vitamin D is mainly synthesized in the skin after exposure to sunlight, and is produced in only a small amount through foods.

As the percentage of time people spend indoors is gradually increasing, people usually have fewer opportunities for sufficient vitamin D synthesis due to a lack of sun exposure, and accordingly, recently, there are a variety of products including light sources for vitamin D synthesis.

However, most of products for vitamin D synthesis that have been on the market to date are designed to mount on a user's body, and since it takes time to mount on the body, in many cases, users stop using the products due to the hassle.

Additionally, the existing products for vitamin D synthesis are used without considering an amount of vitamin D synthesis actually required for users, and the users have difficulties in setting how long they should use the corresponding products.

Accordingly, there is a need for methods for solving the problems.

### SUMMARY

The present disclosure is designed to solve the above-described problems, and therefore an object of the present disclosure is to improve a user's health by determining an amount of vitamin D synthesis actually required for the user and inducing the synthesis of vitamin D in the corresponding amount.

Another object of the present disclosure is to enable additional synthesis of a required amount of vitamin D for a user in a straightforward manner without making time for vitamin D synthesis or vexing activities such as mounting a device for vitamin D synthesis on the body.

The problems of the present disclosure are not limited to the above-mentioned problems, and these and other problems will be clearly understood by those skilled in the art from the following description.

To achieve the above-described objects, a bathroom system for vitamin D synthesis according to the present disclosure includes an environment information collection unit configured to collect outdoor or external environment information; a user information collection unit configured to collect user activity information associated with an outdoor activity of a user; a data analysis unit configured to derive an estimated amount of vitamin D synthesized in the user for a certain period of time by analyzing the external environment information and the user activity information, and derive an additional amount of vitamin D synthesis additionally required for the user by comparing the estimated amount of synthesized vitamin D with a preset reference recommended amount of vitamin D; a light source for vitamin synthesis provided in a bathroom facility to emit light for vitamin D synthesis in a body of the user; and a light source control unit configured to control the light source for vitamin synthesis according to the additional amount of vitamin D synthesis derived from the data analysis unit.

In this instance, the external environment information collected by the environment information collection unit may include at least one of date data, weather data, ultraviolet (UV) index data, temperature data or fine dust data.

Additionally, the user activity information collected by the user information collection unit may include at least one of movement path data and indoor/outdoor location ratio data of the user or body temperature data of the user.

Furthermore, the light source for vitamin synthesis may be configured to emit UV-B to synthesize vitamin D in the body of the user.

Here, the light source for vitamin synthesis may be configured to emit the UV-B having a wavelength of 280 nm to 320 nm to synthesize vitamin D in the body of the user.

Meanwhile, the light source for vitamin synthesis may be provided in a shower chair on which the body of the user is placed in the bathroom facility.

In this instance, the light source for vitamin synthesis may be provided in a grip of a shower designed for the user to grip in the bathroom facility.

Additionally, the light source control unit may be configured to control at least one of an amount, duration or pattern of light emission of the light source for vitamin synthesis according to the additional amount of vitamin D synthesis.

In addition, to achieve the above-described objects, a method for operating a bathroom system for vitamin D synthesis according to the present disclosure includes (a) collecting outdoor or external environment information through an environment information collection unit; (b) collecting user activity information associated with an outdoor activity of a user through a user information collection unit; (c) deriving, by a data analysis unit, an estimated amount of vitamin D synthesized in the user for a certain period of time by analyzing the external environment information and the user activity information; (d) deriving, by the data analysis unit, an additional amount of vitamin D synthesis additionally required for the user by comparing the estimated amount of synthesized vitamin D derived by the step (c) with a preset reference recommended amount of vitamin ID, and (e) controlling, by a light source control unit, a light source for vitamin synthesis provided in a bathroom facility to emit light for vitamin D synthesis in a body of the user according to the additional amount of vitamin D synthesis derived in the step (d).

In this instance, the step (e) may include controlling, by the light source control unit, a wavelength of the light source for vitamin synthesis between 280 nm and 320 nm, considering the additional amount of vitamin D synthesis.

Additionally, the step (e) may include, controlling, by the light source control unit, at least one of an amount, duration or pattern of light emission of the light source for vitamin synthesis, considering the additional amount of vitamin D synthesis.

The bathroom system for vitamin D synthesis of the present disclosure to solve the above-described problems and its operating method improves the user's health by deriving the estimated amount of vitamin D synthesized in the user for a certain period of time by analysis of the external environment information and the user activity information respectively acquired from the environment information collection unit and the user information collection unit, and deriving the additional amount of vitamin D synthesis required for the user P using the estimated amount of synthesized vitamin D to induce the synthesis of vitamin D in the actually required amount for the user P.

Additionally, with the light source for vitamin synthesis provided in the bathroom facilities, the present disclosure enables additional synthesis of the required amount of vitamin D for the user in a straightforward manner through daily activities essentially done every day without making time for vitamin D synthesis or vexing activities such as mounting a device for vitamin D synthesis on the body.

The effects of the present disclosure are not limited to the above-mentioned effects, and these and other effects will be clearly understood by those skilled in the art from the appended claims.

### BRIEF DESCRIPTION OF THE DRAWINGS

FIG. 1 is a diagram schematically showing each component of a bathroom system for vitamin D synthesis according to an embodiment of the present disclosure.

FIG. 2 is a diagram showing the type of external environment information and user activity information respectively acquired from an environment information collection unit and a user information collection unit in a bathroom system for vitamin D synthesis according to an embodiment of the present disclosure.

FIG. 3 is a diagram sequentially showing each step of a method for operating a bathroom system for vitamin D synthesis according to an embodiment of the present disclosure.

FIG. 4 is a diagram showing an example of bathroom facilities equipped with a light source for vitamin synthesis in a bathroom system for vitamin D synthesis according to an embodiment of the present disclosure.

### DETAILED DESCRIPTION

Hereinafter, exemplary embodiments of the present disclosure for achieving the objects of the present disclosure will be described with reference to the accompanying drawings. In the description of this embodiment, the same name and reference sign is used for the same element and its detailed description is omitted.

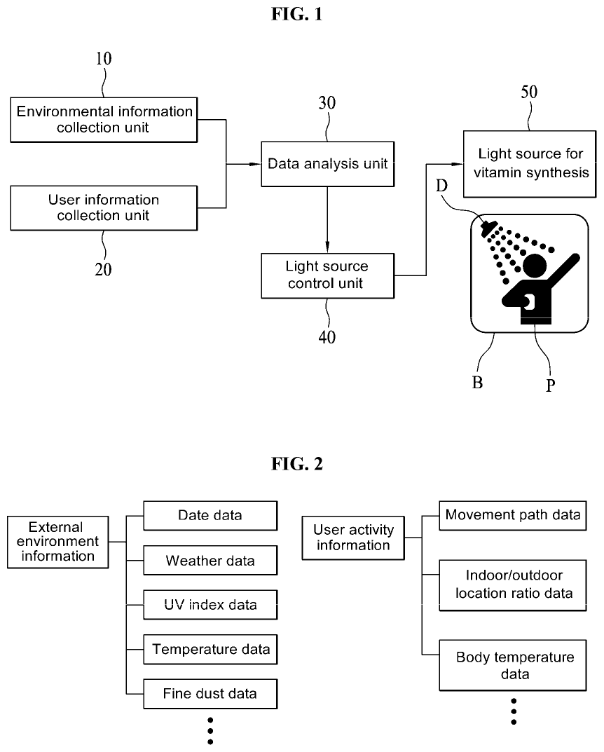

FIG. 1 is a diagram schematically showing each component of a bathroom system for vitamin D synthesis according to an embodiment of the present disclosure.

As shown in FIG. 1, the bathroom system for vitamin D synthesis according to an embodiment of the present disclosure includes an environment information collection unit 10, a user information collection unit 20, a data analysis unit 30, a light source 50 for vitamin synthesis and a light source control unit 40.

The environment information collection unit 10 collects outdoor or external environment information, and the user information collection unit 20 collects user activity information associated with an outdoor activity of a user P.

The environment information collection unit 10 and the user information collection unit 20 are the components that collect information used to derive an estimated amount of vitamin D synthesized in the user.

In this instance, as shown in FIG. 2, the external environment information collected by the environment information collection unit 10 may include at least one of date data, weather data, ultraviolet (UV) index data, temperature data or fine dust data.

Additionally, the user activity information collected by the user information collection unit 20 may include at least one of movement path data and indoor/outdoor location ratio data of the user P or body temperature data of the user P.

That is, the environment information collection unit 10 collects the outdoor or external environment information to derive how much vitamin D can be synthesized in the current external environment condition, and the user information collection unit 20 collects the outdoor activity information and body information of the user P to determine how much the user P is exposed to the external environment.

To this end, the environment information collection unit 10 may include various types of sensors to measure various outdoor environment conditions, and the user information collection unit 20 may include devices such as user terminals or wearable devices to measure the movement path and body information of the user P.

The data analysis unit 30 plays a role in deriving the estimated amount of vitamin D synthesized in the user P for a certain period of time by analyzing the external environment information and the user activity information collected as described above, and deriving an additional amount of vitamin D synthesis additionally required for the user P by comparing the estimated amount of synthesized vitamin D with a preset reference recommended amount of vitamin D.

That is, the data analysis unit 30 estimates an amount of vitamin D per unit time synthesized in the body of the user P through the collected external environment information and the collected user activity information, and estimates the estimated amount of vitamin D synthesized in the user P for a set period of time by multiplying the amount of synthesized vitamin D per unit time by the corresponding period of time.

Additionally, the data analysis unit 30 determines if the derived estimated amount of synthesized vitamin D meets the preset reference recommended amount of vitamin D by comparing the estimated amount of synthesized vitamin D with the reference recommended amount of vitamin D, i.e., the reference recommended amount of vitamin D generally required considering the age, body type and gender of the user P.

When it is determined that the derived estimated amount of synthesized vitamin D does not meet the reference recommended amount of vitamin D, the data analysis unit 30 derives the additional amount of vitamin D synthesis additionally required for the user P.

The light source 50 for vitamin synthesis is the component provided in facilities D in the bathroom B to emit light for vitamin D synthesis in the body of the user P.

The light source 50 for vitamin synthesis includes any light source configured to emit UV-B to synthesize vitamin D in the user's body, for example, a light-emitting diode (LED).

In this instance, the light source 50 for vitamin synthesis is configured to emit UV-B having the wavelength of 280 nm to 320 nm to create an optimal environment for vitamin D synthesis.

The light source control unit 40 plays a role in controlling the light source for vitamin synthesis according to the additional amount of vitamin D synthesis derived from the data analysis unit 30.

Specifically, the light source control unit 40 is configured to control at least one of the amount, duration or pattern of light emission of the light source 50 for vitamin synthesis to enable the synthesis of vitamin D in the required additional amount of vitamin D synthesis.

Hereinafter, a process of operating the bathroom system for vitamin D synthesis including the above-described components will be described.

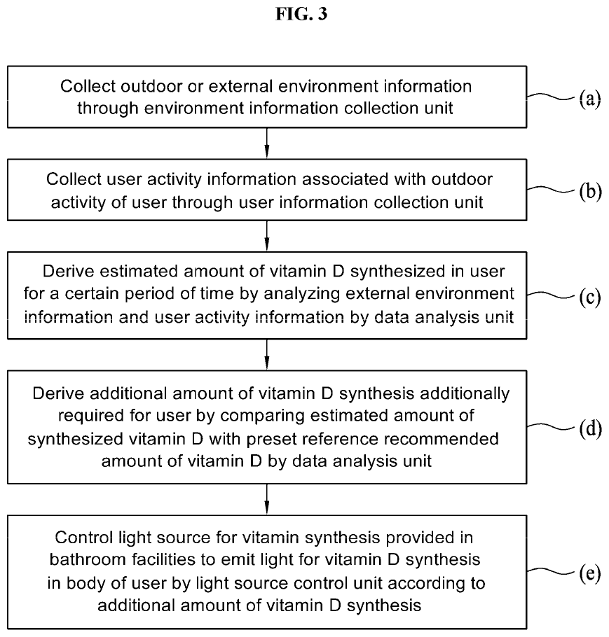

FIG. 3 is a diagram sequentially showing each step of a method for operating the bathroom system for vitamin D synthesis according to an embodiment of the present disclosure.

As shown in FIG. 3, the method for operating the bathroom system for vitamin D synthesis according to an embodiment of the present disclosure includes (a) collecting the outdoor or external environment information through the environment information collection unit 10, (b) collecting the user activity information associated with the outdoor activity of the user P through the user information collection unit 20, (c) deriving, by the data analysis unit 30, the estimated amount of vitamin D synthesized in the user P for the certain period of time by analyzing the external environment information and the user activity information, (d) deriving, by the data analysis unit 30, the additional amount of vitamin D synthesis additionally required for the user P by comparing the estimated amount of synthesized vitamin D derived by the step (c) with the preset reference recommended amount of vitamin D, and (e) controlling, by the light source control unit 40, the light source 50 for vitamin synthesis provided in the facilities D in the bathroom B to emit light for vitamin D synthesis in the body of the user P according to the additional amount of vitamin D synthesis derived in the step (d).

That is, the step (a) includes collecting, by the environment information collection unit 10, the external environment information including at least one of date data, weather data, UV index data, temperature data or fine dust data, and the step (b) includes collecting, by the user information collection unit 20, the user activity information including at least one of the movement path data and indoor/outdoor location ratio data of the user P or the body temperature data of the user P.

Subsequently, the step (c) includes estimating, by the data analysis unit the amount of vitamin D per unit time synthesized in the body of the user P through the collected external environment information and the collected user activity information, and estimating the estimated amount of vitamin D synthesized in the user P for the arbitrarily set period of time by multiplying the amount of synthesized vitamin D per unit time by the corresponding period of time.

Additionally, the step (d) includes determining, by the data analysis unit if the derived estimated amount of synthesized vitamin D estimated by the step (c) meets the preset reference recommended amount of vitamin D by comparing the estimated amount of synthesized vitamin D with the reference recommended amount of vitamin D.

When it is determined that the derived estimated amount of synthesized vitamin D does not meet the reference recommended amount of vitamin D, the data analysis unit 30 derives the additional amount of vitamin D synthesis additionally required for the user P.

Additionally, the step (e) includes controlling at least one of the amount, duration or pattern of light emission of the light source 50 for vitamin synthesis according to the additional amount of vitamin D synthesis derived in the step (d) to enable the synthesis of vitamin D in the additional amount of vitamin D synthesis required for the user P.

In this instance, the step (e) may include controlling, by the light source control unit 40, the wavelength of the light source 50 for vitamin synthesis between 280 nm and 320 nm, considering the additional amount of vitamin D synthesis.

As described above, the present disclosure derives the estimated amount of vitamin D synthesized in the user for the certain period of time by analyzing the external environment information and the user activity information respectively acquired from the environment information collection unit 10 and the user information collection unit 20, and derives the additional amount of vitamin D synthesis additionally required for the user P using the estimated amount of synthesized vitamin D to induce the synthesis of vitamin D in the actually required amount for the user P in order to improve the health of the user P.

Meanwhile, with the light source 50 for vitamin synthesis provided in the facilities D in the bathroom B, the present disclosure enables additional synthesis of the required amount of vitamin D in the user P in a straightforward manner through daily activities essentially done every day without making time for vitamin D synthesis or vexing activities such as mounting a device for vitamin D synthesis on the body.

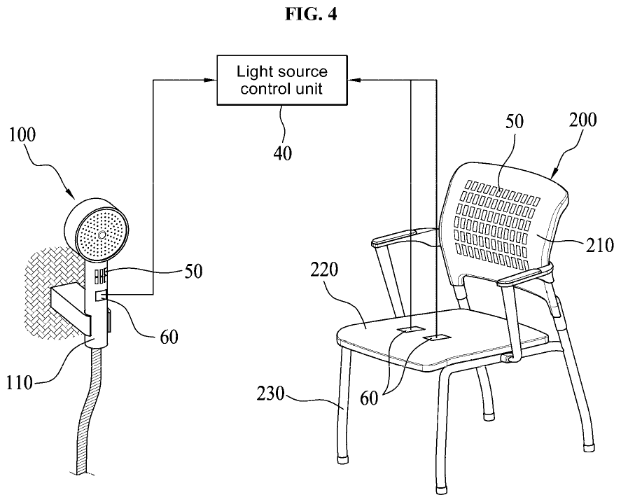

FIG. 4 is a diagram showing an example of the facilities D in the bathroom B equipped with the light source 50 for vitamin synthesis in the bathroom system for vitamin D synthesis according to an embodiment of the present disclosure.

As shown in FIG. 4, the facilities D in the bathroom B equipped with the light source 50 for vitamin synthesis may be facilities that directly contact or face the body while the user P is using the bathroom B, such as a shower 100 and a shower chair 200.

In the illustrated example, in the case of the shower 100, the light source 50 for vitamin synthesis is provided in a grip 110 that the user P grips by the hand, and in the case of the shower chair 200, the light source 50 for vitamin synthesis is provided in a backrest 210 on which the user's back is placed.

However, this is provided by way of illustration, and the light source 50 for vitamin synthesis may be provided in the shower head of the shower 100, and may be provided in a seat 220 or a support leg 230 of the shower chair 200.

Additionally, the shower 100 and the shower chair 200 may be provided in an integrated form. For example, they may be integrally provided such that the shower 100 and a faucet that supplies water to the shower are mounted in the rear surface of the backrest 210 of the shower chair 200, thereby providing a combined effect.

Meanwhile, in the facilities D in the bathroom B equipped with the light source 50 for vitamin synthesis, a body detection sensor 60 may be further provided to detect the user's body to determine if the user P is using the corresponding facilities D.

In the illustrated example, in the case of the shower 100, the body detection sensor 60 is additionally provided in the grip 110 that the user P grips by the hand, and in the case of the shower chair 200, the body detection sensor 60 is provided in the seat 220 on which the user's hip is placed.

That is, when the user uses the corresponding facilities D, the body detection sensor 60 detects the user's body and transmits detection data to the light source control unit 40, and the light source control unit 40 controls the light source 50 for vitamin synthesis upon receiving the detection data, thereby preventing the light source 50 for vitamin synthesis from emitting light at unnecessary times.

In this instance, the body detection sensor 60 applied to the grip 110 of the shower 100 may include a variety of sensors that can be suitably applied to the shower 100, including an optical sensor and a button sensor, and the body detection sensor 60 provided in the seat 220 of the shower chair 200 may include a variety of sensors that can be suitably applied to the shower chair 200, including a weight sensor, an optical sensor and a touch sensor.

The exemplary embodiments according to the present disclosure have been hereinabove described, and it is obvious to those skilled in the art that the present disclosure is not limited to the disclosed embodiments and may be embodied in other particular forms without departing from the scope or spirit of the present disclosure. Therefore, the above-described embodiments should be considered as exemplary, not limiting, and accordingly, the present disclosure is not limited to the foregoing description and changes may be made thereto within the scope of the appended claims and its equivalent scope.

1. A bathroom system for vitamin D synthesis, comprising:
an environment information collection unit configured to collect outdoor or external environment information;
a user information collection unit configured to collect user activity information associated with an outdoor activity of a user;
a data analysis unit configured to derive an estimated amount of vitamin D synthesized in the user for a certain period of time by analyzing the external environment information and the user activity information, and derive an additional amount of vitamin D synthesis additionally required for the user by comparing the estimated amount of synthesized vitamin D with a preset reference recommended amount of vitamin D;
a light source for vitamin synthesis provided in a facility in a bathroom to emit light for vitamin D synthesis in a body of the user; and
a light source control unit configured to control the light source for vitamin synthesis according to the additional amount of vitamin D synthesis derived from the data analysis unit.

2. The bathroom system for vitamin D synthesis according to claim 1, wherein the external environment information collected by the environment information collection unit includes at least one of date data, weather data, ultraviolet (UV) index data, temperature data or fine dust data.

3. The bathroom system for vitamin D synthesis according to claim 1, wherein the user activity information collected by the user information collection unit includes at least one of movement path data and indoor/outdoor location ratio data of the user or body temperature data of the user.

4. The bathroom system for vitamin D synthesis according to claim 1, wherein the light source for vitamin synthesis is configured to emit UV-B to synthesize vitamin D in the body of the user.

5. The bathroom system for vitamin D synthesis according to claim 4, wherein the light source for vitamin synthesis is configured to emit the UV-B having a wavelength of 280 nm to 320 nm to synthesize vitamin D in the body of the user.

6. The bathroom system for vitamin D synthesis according to claim 1, wherein the light source for vitamin synthesis is provided in a shower chair on which the body of the user is placed in the facility in the bathroom.

7. The bathroom system for vitamin D synthesis according to claim 1, wherein the light source for vitamin synthesis is provided in a grip of a shower designed for the user to grip in the facility in the bathroom.

8. The bathroom system for vitamin D synthesis according to claim 1, wherein the light source control unit is configured to control at least one of an amount, duration or pattern of light emission of the light source for vitamin synthesis according to the additional amount of vitamin D synthesis.

9. A method for operating a bathroom system for vitamin D synthesis, comprising:
(a) collecting outdoor or external environment information through an environment information collection unit;
(b) collecting user activity information associated with an outdoor activity of a user through a user information collection unit;
(c) deriving, by a data analysis unit, an estimated amount of vitamin D synthesized in the user for a certain period of time by analyzing the external environment information and the user activity information;
(d) deriving, by the data analysis unit, an additional amount of vitamin D synthesis additionally required for the user by comparing the estimated amount of synthesized vitamin D derived by the step (c) with a preset reference recommended amount of vitamin D; and
(e) controlling, by a light source control unit, a light source for vitamin synthesis provided in a facility in a bathroom to emit light for vitamin D synthesis in a body of the user according to the additional amount of vitamin D synthesis derived in the step (d).

10. The method for operating a bathroom system for vitamin D synthesis according to claim 9, wherein the step comprises controlling, by the light source control unit, a wavelength of the light source for vitamin synthesis between 280 nm and 320 nm, considering the additional amount of vitamin D synthesis.

11. The method for operating a bathroom system for vitamin D synthesis according to claim 9, wherein the step (e) comprises, controlling, by the light source control unit, at least one of an amount, duration or pattern of light emission of the light source for vitamin synthesis, considering the additional amount of vitamin D synthesis.
